(19) United States
(12) Patent Application Publication
(10) Pub. No.: US 2003/0191514 A1
(43) Pub. Date: Oct. 9, 2003

(54) Heated personal boating blanket

(76) Inventor: Richard Scott Chatham, Auburn, WA (US)

Correspondence Address: Richard Scott Chatham, 22421 SE 331st ST, Auburn, WA, 98092, US

(21) Appl. No.: 10/115,708

(22) Filed: Apr. 3, 2002

(51) Int. Cl. A61F 7/00

(52) U.S. Cl. 607/107

(57) ABSTRACT

A heated personal boating blanket having a wind and water resistant nylon covering and woven fleece lining for delivering thermally controlled air to a occupants in a boat. The nylon covering and fleece lining are connected at the periphery forming an inflatable chamber into which a heated air supply hose from the boat heater is inserted to supply heated air to the inflatable chamber. The heated air is dissipated from the inflatable chamber through the fleece lining directly onto the occupants draped in the heated personal boating blanket. Utilizing the existing flexible boat heater hose as a source of heat there is no need for bulky external heating apparatus. Utilizing the existing flexible boat heater hose eliminates the possibility of electrical shock from internally electrically heated blankets. The heated personal boating blanket does not attach to boat occupants in any way thus avoiding possible entanglement of the boat occupants in emergency situations and upsets.

### CROSS-REFERENCE TO RELATED APPLICATIONS

Not Applicable

### STATEMENT REGARDING FEDERALLY SPONSERED RESEARCH OR DEVELOPMENT

Not Applicable

### REFERENCE TO SEQUENCE LISTING; A TABLE OR COMPUTER PROGRAM LISTING COMPACT DISK APPENDIX

Not Applicable

### FIELD OF THE INVENTION

The field of the invention relates to blankets, more particularly to blankets having thermal heating from external sources.

### BACKGROUND OF THE INVENTION

The nature and use of thermal blankets is well known. The prior art comprises various types and styles of blankets intended for any number of uses. These include uses as bedclothes, for use in emergency situations in order to warm or cool a victim and for covering. All these require auxiliary heating apparatus or utilize internal electrical heating elements. What is needed if a blanket for boat occupants having a safe and readily available source of heat. What is needed is a blanket which provides thermal heating without endangering the occupants to electrical shock. What is needed is a blanket that will not impede the occupants exit from the boat in the case of emergency or upset. What is needed is a blanket that warms and dries occupants upon exit from the water. What is needed is a blanket that protects boat occupants from wind chill in speeding boats. The present invention meets these needs and other needs.

### BRIEF SUMMARY OF THE INVENTION

In accordance with the objectives of this invention described above and to overcome the limitations of the prior art, a heated personal boating blanket is provided for convectively warming boat occupants. The boat blanket comprises a nylon covering and fleece lining connected at the periphery forming an inflatable chamber into which a heated air supply hose from the boat heater is inserted to supply heated air to the inflatable chamber. The heated air is dissipated through the woven fleece lining directly onto the occupants draped in the personal heated boating blanket.

### BRIEF DESCRIPTION OF THE SEVERAL VIEWS OF THE DRAWING

For a more complete understanding of this invention reference is now made to the following detailed description of the embodiments as illustrated in the accompanying drawing, wherein:

FIG. 1 is a side perspective view of a sitting boat occupant and a heated personal boating blanket according to the invention with lead lines illustrating how the blanket would be received by a sitting boat occupants, and also illustrating a boat heater flexible hose supplying heated air to the boating blanket.

FIG. 2 is a bottom view illustrating the bottom of the boating blanket and the foot receptacle.

FIG. 3 is a top plan view of a heated personal boating blanket for boat occupants.

### DETAIL DESCRIPTION OF THE INVENTION

This invention is described in the following description with reference to the figures, in which like numbers represents the same or similar elements. Variations from this description may be accomplished without deviating from the spirit or scope of the invention.

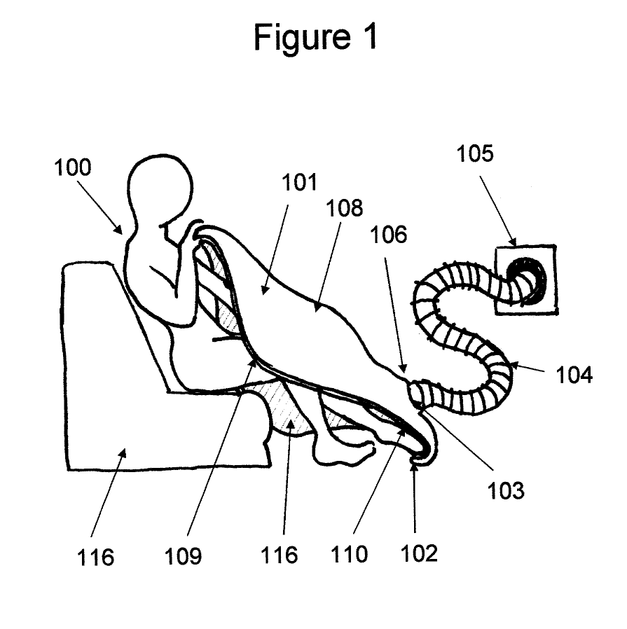

FIG. 1 illustrates a boat occupant 100 in a sitting position on a boat seat 116. The occupant may be in a variety of positions within the boat. The occupant is illustrated in the sitting position with thighs generally perpendicular to the boat deck, lower legs extending downward to the boat deck and feet resting upon the deck. A heated personal boating blanket indicated by reference numeral 101 is shown with connecting lines that illustrate how it would be draped over the boat occupant. The heated personal boating blanket includes a foot receptacle 102 which in addition to providing heat to the occupants' feet also serves as an anchor to keep the blanket in position. The foot receptacle can be turned upside down and used as a hood for the occupants' head.

The heated personal boating blanket includes an inlet 103 that receives warmed air to the inflatable chamber. The warmed air is provided by an air hose 104 from the forced air boat heating unit 105. The boat-heating unit includes user-selectable fan speeds, controllable heat amounts and temperature control. The inlet in the inflatable section is lined with a hook and loop closure 106 to receive and retain the end of the heater hose. Using this configuration forced and heated air can flow through the air hose into the inflatable chamber 107.

The heated personal boating blanket of this invention may be constructed using methods and materials that are known for making similar products. The heated personal boating blanket is assembled from a base sheet of non-pile fleece 115 which is the bottom. The top layer compromising a sheet of synthetic material having wind and water resistant qualities 108. For example, the top may be nylon material with PVC backing to provide weather proofing.

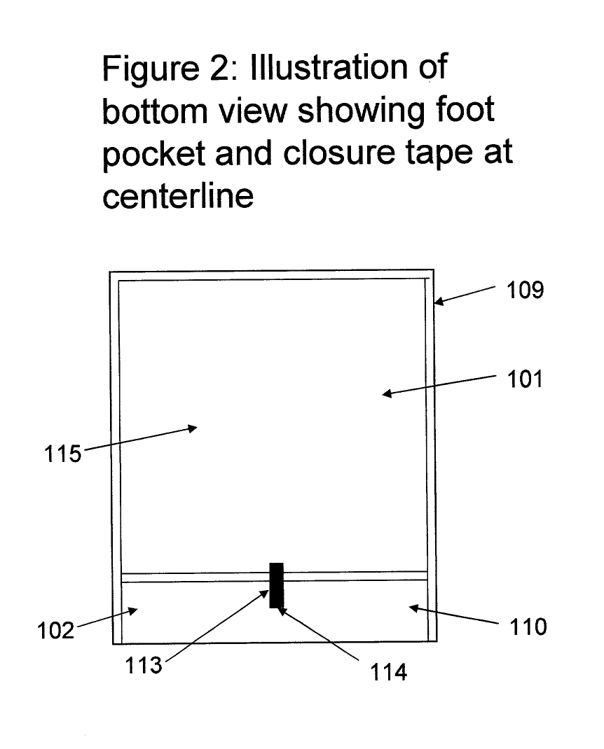

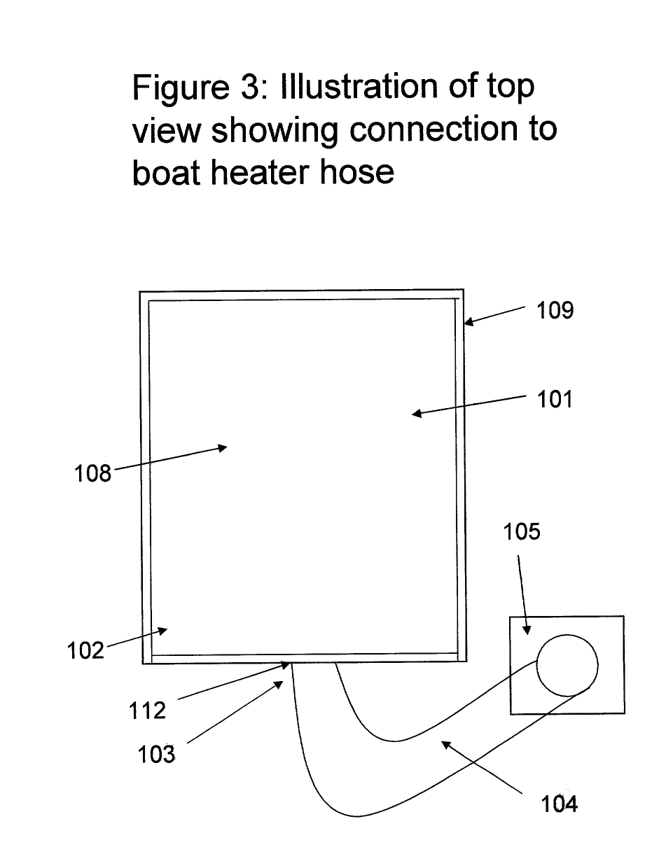

FIG. 2 shows the top and bottom layers attached at the top and side periphery utilizing a nylon seam tape 109 and machine sown. The bottom periphery of the top layer is attached to heavy PVC nylon several inches from the bottom of the heated personal boating blanket 110. The PVC nylon is then turned under to form the foot pocket 111. Forming of the foot pocket is accomplished by attaching the left and right side of the blanket utilizing nylon seam tape and machine sewn. An inlet 103 is provided at the junction of the top nylon layer and the heavy PVC nylon extending several inches either side of the center line. The inlet is lined with hook and loop nylon 106 to facilitate the heater hose 104 entry and to permit a snug fit around the heater hose for retention. A small pointed tab 112 is attached to each side of the inlet to facilitate ease of opening and closing. FIG. 3 shows a strip of nylon loop tape 113 attached to the inside of the foot pocket 102 and when folded over the top edge of the foot pocket contacts a strip of nylon hook tape 114 attached to the exterior side of the foot pocket which holds the center section of the foot pocket in place.

I claim as my invention:

1. An inflatable heated boating blanket for convectively warming boat occupants. An inflatable covering including an upper sheet and lower woven sheet sown together at the periphery forming an inflatable chamber, the woven base sheet allowing heated air to escape directly onto the boat occupants draped under the boat blanket.

2. An inflatable heated boating blanket includes a foot pocket at the base of the blanket allowing for the boat occupants feet to be inserted in the pocket and warmed by escaping heated air from the woven base sheet.
A foot pocket formed at the base of the heated blanket which acts as an anchor for the blanket preventing the blanket from rising in the wind or when the boat is operating at higher speeds:
A foot pocket formed at the base of the heated blanket which can be rotated 180 degrees and become a hood for heating boat occupants heads when draped under the heated blanket.

3. An inflatable heated personal boating blanket that warms and dries boat occupants exiting from the water.

4. An inflatable heated personal boating blanket that utilizes existing flexible boat heater hose as a source of heat.
An inflatable heated personal boating blanket that does not entangle the boat occupants in case of emergency or upset.
An inflatable heated personal boating blanket which is not attached to boat occupants in any manner except for the occupants' feet inserted into a loosely fitted pocket and allows for ease of extraction in case of emergency or upset.
